(19) United States
(12) Patent Application Publication
(10) Pub. No.: US 2006/0064043 A1
(43) Pub. Date: Mar. 23, 2006

(54) Method and device for determining an articulation point of two bones

(75) Inventors: Andreas Goeggelmann, Ingersheim (DE); Josef Kozak, Tuttlingen (DE); Hanns-Peter Tuemmler, Tuttlingen (DE)

Correspondence Address: Lipsitz & McAllister, LLC, 755 MAIN STREET, MONROE, CT, 06468, US

(73) Assignee: AESCULAP AG & Co. KG, Tuttlingen (DE)

(21) Appl. No.: 11/234,863

(22) Filed: Sep. 23, 2005

Related U.S. Application Data
(63) Continuation of application No. PCT/EP04/01665, filed on Feb. 20, 2004.

(30) Foreign Application Priority Data
Mar. 27, 2003 (DE) 103 13 747.5

(51) Int. Cl. A61B 5/11

(52) U.S. Cl. 600/595

(57) ABSTRACT

In order to improve the precision of determining the articulation point with a method for determining an articulation point of two bones, which are connected by a joint and are connected to pivot relative to one another around at least two non-parallel rotational axes, wherein the two bones are moved relative to one another, measured values describing the movement of a point of a bone in a reference system of the other bone are recorded, a rotational axis describing the movement as rotation is determined from these measured values for a plurality of parts of the path or respectively for one of a plurality of consecutively recorded paths, it is proposed that the deviation in orientation is calculated for the differently oriented rotational axes determined in this way and from the differently oriented rotational axes an articulation point is calculated only when the deviation in orientation of the rotational axes exceeds a specific value.

This application is a continuation of international application number PCT/EP2004/001665 of Feb. 20, 2004.

The present disclosure relates to the subject matter disclosed in international patent application PCT/EP2004/001665 of Feb. 20, 2004 and German application number 103 13 747.5 of Mar. 27, 2003, which are incorporated herein by reference in its entirety and for all purposes.

### BACKGROUND OF THE INVENTION

The invention relates to a method for determining an articulation point of two bones, which are connected by a joint and are connected to pivot relative to one another around at least two non-parallel rotational axes, wherein the two bones are moved relative to one another, measured values describing the movement of a point of a bone in a reference system of the other bone are recorded, a rotational axis describing the movement as rotation is determined from these measured values for a plurality of parts of the path or respectively for one of a plurality of consecutively recorded paths.

Under specific conditions the position of anatomical articulation points can be determined by moving the bones pivoted to one another at these articulation points relative to one another and through analysis of the movement, e.g. by means of a navigation system, calculating the position of the articulation point from the recorded path curves. This is possible without any problem, for example, with joints structured as ball-and-socket joints, as is the case with the hip joint. Such a method is described, inter alia, in EP 0 969 780 B1.

However, it is more difficult to perform the exact determination of an articulation point for those joints that are not structured as pure ball-and-socket joints, but are essentially hinge joints or hinge-type joints. When a joint is a pure hinge joint, there is a fixed rotational axis, but no central articulation point. Therefore, there is no sense in determining such an articulation point in such joints.

However, most hinge joints or hinge-type joints in the body of a mammal are structured so that they can not only be pivoted around a single rotational axis, but so that it is also possible—at least to a limited extent—to pivot around a second rotational axis. This is the case with the knee joint, for example. Besides the normal pivoting movement around the transverse axis, the tibia can be pivoted to a small degree around its longitudinal axis, therefore there exist essentially two mutually perpendicular rotational axes. These rotational axes jointly determine an articulation point for the knee, around which every movement occurs, even when it is formed from the rotations around the two described rotational axes as a superposed movement. An articulation point can therefore be determined in these cases, although it is necessary to actually also pivot both bones relative to one another around these two rotational axes to be able to determine this articulation point. A pivoting movement alone around one of the two rotational axes would not be sufficient, since determination of one rotational axis alone does not result in definition of an articulation point along this rotational axis.

### SUMMARY OF THE INVENTION

It is an object of the invention to conduct a method of this type so that an articulation point can be determined with the desired accuracy with the movement of two bones.

This object is achieved according to the invention with a method of the above-described type in that the deviation in orientation is calculated for the differently oriented rotational axes determined in this way and from the differently oriented rotational axes an articulation point is calculated only when the deviation in orientation of the rotational axes exceeds a specific value.

The invention therefore is based on the recognition that all actually determined rotational axes are formed by superposition of the rotational axes specific to the joint, i.e. in the case of the knee, for example, as a superposition of the flexing axis of the knee, on the one hand, and the longitudinal axis of the tibia as second rotational axis, on the other. The deviation of this orientation is a measure for how strongly the components of these two main rotational axes are represented in the differently oriented rotational axes. If the joint is only moved so that all rotational axes are substantially parallel, then it is impossible to determine an articulation point. The more strongly the differently oriented rotational axes deviate from one another in their orientation, the more accurately they define the sought articulation point through their intersection.

Therefore, measurements, in which the rotational axes differ from one another too little in their orientation, i.e. in which an exact determination of position of the articulation point is not possible, can be eliminated by determining the deviation. Conversely, reliable values for the articulation point are calculated when the different orientations of the rotational axes deviate sufficiently from one another and thus have a high divergence.

Therefore, when recording the movement it is not necessary to ensure that the movement of the two bones is performed precisely around the two main axes, but it is sufficient if the deviation in orientation of the recorded rotational axes is large enough to be sure that components of both main rotational axes have been adequately taken into consideration. Therefore, the tibia can be moved in any desired way relative to the femur, for example, wherein the degrees of freedom provided by the knee are utilised, and once a sufficient deviation has resulted therein, it is assured that a reliable determination of the articulation point can be conducted.

In principle, a wide variety of methods can be used to determine differently oriented rotational axes from the path curves of the two bones moved relative to one another.

In a particularly preferred method, it is provided that during a movement of the two bones relative to one another in different relative positions the instantaneous orientation of one bone in the reference system of the other bone is determined and for this instantaneous orientation an instantaneous rotational axis is calculated, which corresponds to a rotation of the bone from an assumed reference orientation into this instantaneous orientation, and that the deviation in orientation is determined as a result of the deviation of the instantaneous rotational axes.

It is advantageous if the deviation in orientation of the executed movement is represented on a display. It can then be seen immediately on the display whether the deviation in orientation of the rotational axes is sufficiently large, and if this should not be the case, it can be ensured that the deviation reaches the desired value by changing the course of movement.

It is favourable if the determination of an articulation point is blocked as long as the specific value of the deviation in orientation has not been reached. In this way, one is confident of not conducting an erroneous determination of the articulation point.

It is advantageous if to determine the movement of one bone in a reference system of the other bone, the position of the one bone relative to the other is tracked via a navigation system and a marking element is fixed to the one bone. If the other bone is firmly held during the relative movement of the two bones, then the path curve of the one bone in relation to the other can be readily recorded.

The firm hold of the other bone can be dispensed with, if a marking element is also fixed to the other bone.

The method can be conducted particularly advantageously on the knee joint by moving the tibia relative to the femur.

The invention additionally relates to a device for conducting the described method with a navigation system, at least one marking element of the navigation system, which can be fixed to the one bone, and with a data processing unit determining a rotational axis describing the movement as rotation from the recorded measured values for a part of the path or respectively for one of a plurality of consecutively recorded paths.

To also be able to reliably determine an articulation point with such a device in joints, which are not structured as a ball-and-socket joint, it is proposed that the data processing unit calculates the deviation in orientation for the differently oriented rotational axes determined in this way and from the differently oriented rotational axes calculates an articulation point only when the deviation in orientation of the rotational axes exceeds a specific value.

In particular it can be provided that during a movement of the two bones relative to one another in different relative positions the data processing unit determines the instantaneous orientation of one bone in the reference system of the other bone and for this instantaneous orientation calculates an instantaneous rotational axis, which corresponds to a rotation of the bone from an assumed reference orientation into this instantaneous orientation, and that the data processing unit determines the deviation in orientation as a result of the deviation of the instantaneous rotational axes.

It is favourable if the data processing unit has an associated display, on which the deviation in orientation of the executed movement is displayed.

According to a preferred embodiment it is provided that the data processing unit blocks the determination of an articulation point as long as the specific value of the deviation in orientation has not been reached.

The navigation system can have a respective associated marking element both on the one and on the other bone.

### BRIEF DESCRIPTION OF THE DRAWINGS

FIG. 1 is general view of the device for determining the knee articulation point of a patient with a navigation system and a data processing unit;

FIG. 2 is a schematic view of the main rotational axes of a tibia relative to a femur;

FIG. 3 is a schematic view of the knee joint with a bundle of differently oriented knee joint pivot axes; and

FIG. 4 is a schematic view of the recording of different orientations of the bones pivoted relative to one another and the position of a rotational axis that can be derived therefrom.

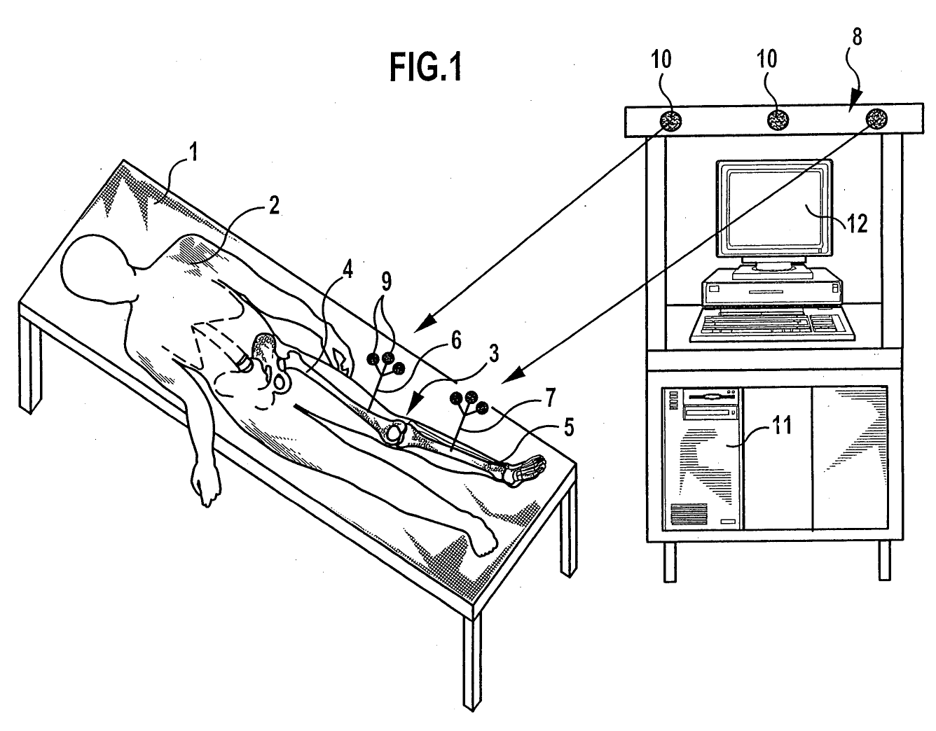

### DETAILED DESCRIPTION

A patient 2, for whom the articulation point of the knee joint 3 is to be determined, lies on an operating table 1. Shown in the drawing are the femur 4 and the tibia 5, which are pivoted to one another via the knee joint 3. A marking element 6 and 7 respectively, which interacts with a navigation system 8 set up in the operating theatre in a known manner, is attached to both the femur 4 and to the tibia 5. The marking elements 6, 7 respectively carry three reference bodies 9 spaced from one another, which either transmit an electromagnetic radiation themselves or reflect an electromagnetic radiation incident on them, e.g. an infrared radiation. This radiation is recorded by three receivers 10 of the navigation system 8 arranged at a distance from one another, and in this way the exact position and orientation of the marking elements 6, 7 can be spatially determined by the navigation system. Data sets corresponding to the respective position are fed from the navigation system 8 to a data processing unit 11, which can represent these data and data derived from these on a display 12.

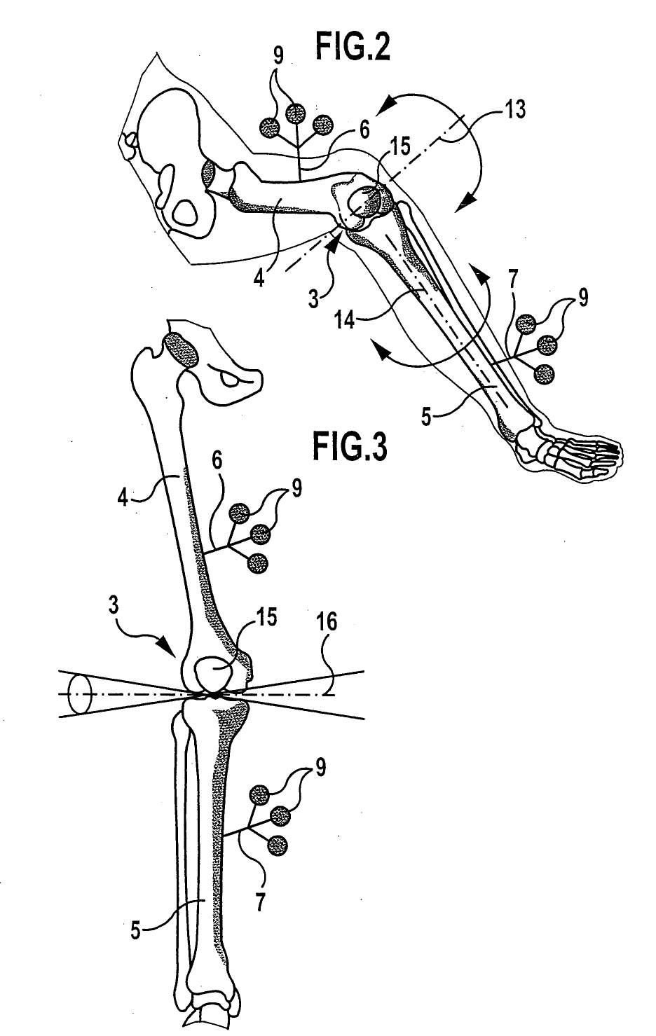

FIG. 2 shows which movements the tibia 5 can execute relative to the femur 4. This movement is substantially a hinge-like pivoting movement around a bending axis 13 running transversely to the longitudinal direction of the femur and tibia and besides this a rotation around the longitudinal axis 14 of the tibia 5. This rotation around the longitudinal axis 14 is limited and only possible over a relatively small angle range, whereas the pivoting around the bending axis 13 can cover an angle of clearly more than 90°.

The articulation point 15 of the knee joint 3 can be determined from the intersection of the bending axis 13 and the longitudinal axis 14. If the two axes do not intersect exactly, but only run very closely past one another, this point can also be defined as a point lying between the two axes and as close to these as possible.

To be able to determine the position of this articulation point 15 solely from the movement of the tibia relative to the femur, the tibia 5 is moved relative to the femur 4 in such a way that this movement no longer consists of only a pure bending of the knee, but also includes components of a rotational movement around the longitudinal axis 14, i.e. a movement of the two bones relative to one another that is as irregular as possible is executed, wherein the person doing the manipulation does not have to be concerned how this movement proceeds precisely.

Since a marking element 6, 7 is respectively fixed to both the femur and to the tibia, the femur 4 can also move at will in this case, the data processing unit can determine the relative movement of the tibia and femur from the movement of the marking element 6 on the femur 4 and the marking element 7 on the tibia 5 and thus the respective position of the marking element 7 and with it of the tibia 5 in a reference system specific to the femur.

From the movement curves of the marking element 7 in the reference system specific to the femur, the rotational axes of this movement can be determined with algorithms known per se. Thus, average rotational axes are determined from the path curves, for example, by averaging different positions and orientations of the marking element 7, and with consecutive movements of the tibia relative to the femur these rotational axes will generally be different, depending on how the tibia had been moved relative to the femur. However, in the case of the knee joint these rotational axes do not normally differ greatly from the bending axis 13, since the movement around this bending axis can cover a substantially larger angle range than the rotation around the longitudinal axis 14. The rotational axes determined in this way then lie, for example, in a double cone 16, the longitudinal axis of which coincides with the bending axis and its tip marks the sought articulation point 15.

This articulation point 15 can be determined only very imprecisely, if the vertex angle of the double cone 16 is very small, i.e. if the deviation of the rotational axes determined in this way from one another is small, however if the angle of the double cone 16 is larger, a more precise determination of the exact position of the articulation point 15 is assured, the larger angle of the double cone 16 corresponding to a larger deviation of the orientation of the rotational axes determined in this way.

The data processing unit 11 determines the deviation or divergence of the orientation values from one another from the individual orientations of the rotational axes and compares these with a given reference value. If the deviation lies below this reference value, the measured values are not used for calculation of an articulation point, since the deviation corresponding to the angle of the double cone 16 is then too low and only imprecise details of the articulation point 15 can be obtained. The articulation point is calculated in the described manner only when the given value of the deviation is exceeded.

The value of the deviation can be represented on the display 22, so that it can be read at any time whether the movement of the tibia relative to the femur has adequately taken into consideration the two pivoting possibilities, i.e. whether this movement was sufficiently irregular. If the deviation threshold is not yet reached, the person doing the manipulation only needs to ensure that upon subsequent movements of the tibia relative to the femur, this movement deviates as strongly as possible from a pure pivoting movement in a single plane.

While it is fundamentally possible to measure the differently oriented rotational axes by consecutive courses of movement of the tibia relative to the femur, it can be provided in another method that during a single course of movement different components of this movement are analysed and used for calculation of a higher number of instantaneous rotational axes.

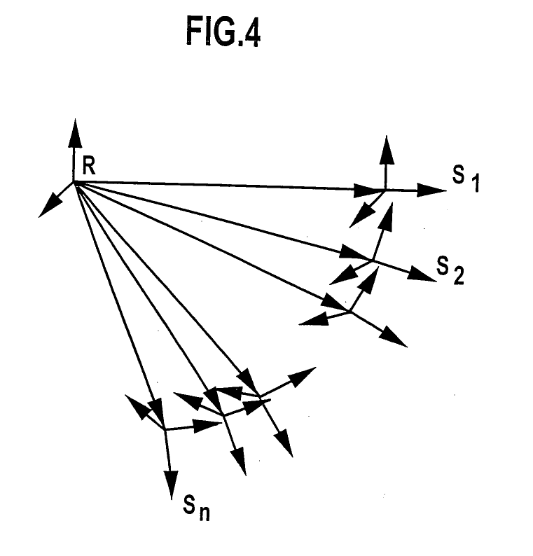

For this, the orientation of the marking element 7 of the tibia 5 in the reference system specific to the femur is respectively determined in different positions during the movement sequence, this is symbolised in FIG. 4 by a plurality of axis crosses, which respectively specify the position of this marking element 7, i.e. with respect to a reference orientation R, which can be calculated, for example, from the mean value of all individual orientations.

For each position of the marking element 7 during the movement sequence, the data processing unit now calculates an instantaneous rotational axis, which is selected so that an axis cross corresponding to a specific position merges into the axis cross of the reference system solely through a rotation around this instantaneous rotational axis. In other words, it is determined around which axis the marking element 7 must be rotated to reach the reference position corresponding to the axis cross R.

This is performed in this manner for all positions of the marking element 7 determined along the path curve and leads to a number of differently oriented instantaneous rotational axes. These instantaneous rotational axes are again examined with respect to their deviation and with a sufficiently large deviation, i.e. when a given threshold is exceeded, the coordinates of the articulation point 15 are calculated, for example, by determination of their intersection or by other mathematical methods.

It is also essential here that the deviation of the different instantaneous orientation forms a criterion for whether such a calculation is relevant or not on the basis of the measurement.

Naturally, it is also possible with this method to repeat the movement of the tibia relative to the femur, but few bending movements and straightening movements of the leg are generally sufficient to obtain an adequate number of measurement points for a very precise determination of the articulation point.

What is claimed is:

1. A method for determining an articulation point of two bones, which are connected by a joint and are connected to pivot relative to one another around at least two non-parallel rotational axes, wherein the two bones are moved relative to one another, measured values describing the movement of a point of one bone in a reference system of the other bone are recorded, a rotational axis describing the movement as rotation is determined from these measured values for a plurality of parts of the path or respectively for one of a plurality of consecutively recorded paths, wherein deviation in orientation is calculated for the differently oriented rotations axes determined in this way and from the differentially oriented rotational axes an articulation pint is calculated only when the deviation in orientation of the rotational axes exceeds a specific value.

2. A method according to claim 1, wherein during a movement of the two bones relative to one another in different relative positions the instantaneous orientation of one bone in the reference system of the other bone is determined and for this instantaneous orientation an instantaneous rotational axis is calculated, which corresponds to a rotation of the bone from an assumed reference orientation into this instantaneous orientation, and the deviation in orientation is determined as a result of the deviation of the instantaneous rotational axes.

3. A method according to claim 1, wherein the deviation in orientation of the executed movement is represented on a display.

4. A method according to claim 1, wherein the determination of an articulation point is blocked as long as the specific value of the deviation in orientation has not been reached.

5. A method according to claim 2, wherein the determination of an articulation point is blocked as long as the specific value of the deviation in orientation has not been reached.

6. A method according to claim 1, wherein to determine the movement of one bone in a reference system of the other bone the position of the one bone relative to the other is tracked by means of a navigation system and a marking element is fixed to the one bone.

7. A method according to claim 6, wherein a marking element is also fixed to the other bone.

8. A method according to claim 1, wherein the one bone is a tibia and the other bone is a femur, which are connected to one another via a knee joint.

9. A device for determining an articulation point of two bones with a navigation system, at least one marking element of the navigation system, which can be fixed to the one bone, and with a data processing unit determining a rotational axis describing the movement as rotation from the recorded measured values for a part of the path or respectively for one of a plurality of consecutively recorded paths, wherein the data processing unit calculates the deviation in orientation of the differently oriented rotational axes determined in this way and from the differently oriented rotational axes calculates an articulation point only when the deviation in orientation of the rotational axes exceeds a specific value.

10. A device according to claim 9, wherein during a movement of the two bones relative to one another in different relative positions the data processing unit determines the instantaneous orientation of one bone in the reference system of the other bone and for this instantaneous orientation calculates an instantaneous rotational axis, which corresponds to a rotation of the bone from an assumed reference orientation into this instantaneous orientation, and that the deviation in orientation is determined as a result of the deviation of the instantaneous rotational axes.

11. A device according to claim 10, wherein the data processing unit has an associated display, on which the data processing unit displays the deviation in orientation of the executed movement.

12. A device according to claim 9, wherein the data processing unit blocks the determination of an articulation point as long as the specific value of the deviation in orientation has not been reached.

13. A device according to claim 9, wherein the navigation system has a respective associated marking element both on the one and on the other bone.
